(19) United States
(12) Patent Application Publication
(10) Pub. No.: US 2024/0024564 A1
(43) Pub. Date: Jan. 25, 2024

(54) SYSTEMS AND METHODS FOR WOUND DRESSING CONNECTOR WITH IMPROVED PRESSURE SENSING

(71) Applicant: KCI Manufacturing Unlimited Company, Westmeath (IE)

(72) Inventor: Christopher Brian Locke, San Antonio, TX (US)

(21) Appl. No.: 18/265,552

(22) PCT Filed: Nov. 16, 2021
(86) PCT No.: PCT/IB2021/060597

§ 371 Date: Jun. 6, 2023

Related U.S. Application Data
(60) Provisional application No. 63/122,748, filed on Dec. 8, 2020.

(51) Int. Cl. A61M 1/00

(52) U.S. Cl. CPC A61M 1/912; A61M 1/966; A61M 2205/7527

(57) ABSTRACT

A new dressing interface such as, for example, a connection pad that includes pressure monitoring at the tissue site is disclosed wherein pressure-detection conduits or lumens are protected from the ingress of wound fluids and exudates by a chamber sealed with a liquid-air separator such as, a hydrophobic filter, positioned within the pad. The liquid-air separator may be bonded in place and sealed with a primary and/or secondary application of an adhesive such as, for example, a UV curable adhesive, that encapsulates the edges of the filter to minimize the amount of liquid from the tissue site seeping into the pressure-detection conduit.

### CROSS-REFERENCE TO RELATED APPLICATIONS

This application claims the benefit of priority to U.S. Provisional Application No. 63/122,748, filed on Dec. 8, 2020, which is incorporated herein by reference in its entirety.

### TECHNICAL FIELD

The invention set forth in the appended claims relates generally to tissue treatment systems and more particularly, but without limitation, to systems and methods including a new dressing interface having a filter for improved pressure-detection performance and a method for assembling the filter within the dressing interface.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems and apparatuses, and methods for improved pressure-detection performance in a negative-pressure therapy environment are set forth in the appended claims. Illustrative embodiments are also provided to enable a person skilled in the art to make and use the claimed subject matter.

For example, in some embodiments, a new dressing interface such as, for example, a connection pad that includes pressure monitoring at the tissue site is disclosed wherein pressure-detection conduits or lumens are protected from the ingress of wound fluids and exudates by a chamber sealed with a liquid-air separator such as, a hydrophobic filter, positioned within the pad. The liquid-air separator may be bonded in place and sealed with a primary and/or secondary application of an adhesive such as, for example, a UV curable adhesive, that encapsulates the edges of the filter to minimize the amount of liquid from the tissue site seeping into the pressure-detection conduit.

In one example embodiment, a dressing interface for connecting a reduced-pressure source to a manifold to treat a tissue site with reduced pressure comprises a conduit housing having a cavity defined by a recessed compartment and an opening, and a base having a an aperture surrounding the opening of the conduit housing. The dressing interface may comprise further a reduced-pressure port extending through the conduit housing and fluidly coupled to the recessed compartment, and adapted to be coupled to the reduced-pressure source. The dressing interface may comprise further a pressure-detection port extending through the conduit housing and adapted to be fluidly coupled to a pressure sensor. A pressure-detection chamber may be fluidly coupled to the pressure-detection port and have an orifice closed by a liquid-air separator to limit the flow of liquids into the pressure-detection chamber.

In a preferred embodiment, the liquid-air separator may be a hydrophobic filter. In some example embodiments, the hydrophobic filter may comprise PTFE. In some embodiments, the hydrophobic filter may comprise a woven fiberglass membrane coated with PTFE. In yet other embodiments, the hydrophobic filter may comprise a woven base cloth. In some embodiments, the dressing interface may further comprise an adhesive disposed on the edge of the orifice between the edges of the hydrophobic filter and the edge of the orifice. In yet other embodiments, the dressing interface may comprise further an adhesive disposed over the edges of the hydrophobic filter and the edge of the orifice to encapsulate the outer edges of the hydrophobic filter. In both cases, the adhesive may comprise a gel curable by UV light.

Alternatively, other example embodiments may be a dressing interface for connecting a reduced-pressure source to a manifold to treat a tissue site with reduced pressure comprises a conduit housing having a cavity defined by a recessed compartment and an opening, and a base having a an aperture surrounding the opening of the conduit housing. The dressing interface may comprise further a reduced-pressure port extending through the conduit housing and fluidly coupled to the recessed compartment, and a pressure-detection port extending through the conduit housing and forming a chamber having an orifice disposed within the recessed compartment. The dressing interface may further comprise a liquid-air separator closing the orifice of the chamber.

The dressing interfaces as described above may be produced by steps associate with application of the adhesive. In one example embodiment, the steps may comprise applying a first bead of adhesive gel to the orifice that is curable by UV light. The steps may further comprise placing the liquid-air separator on the adhesive gel over the orifice to close the chamber, and then curing the adhesive gel with UV light to form a semi-rigid structure when cured by the UV light. In some embodiments, the steps may further comprise applying a second bead of adhesive gel to the liquid-air separator opposite the first bead of adhesive gel over the orifice that is also durable by UV light. The steps may further comprise curing the second bead of adhesive gel with UV light to form another semi-rigid structure when cured by the UV light. In some embodiments, both of the first and second bead of adhesive may be cured simultaneously by the UV light.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is a schematic diagram, in perspective view with a portion in cross-section, of a reduced-pressure treatment system for applying reduced pressure to a tissue site through a dressing interface or a reduced pressure adapter, according to an illustrative embodiment incorporating some of the functionality of the therapy system of FIG. 1;

FIG. 3A is a graph illustrating an illustrative embodiment of pressure control modes for the negative-pressure therapy systems of FIGS. 1 and 2 wherein the x-axis represents time in minutes (min) and/or seconds (sec) and the y-axis represents pressure generated by a pump in Torr (mmHg) that varies with time in a continuous pressure mode and an intermittent pressure mode for applying negative pressure in the therapy system;

FIG. 3B is a graph illustrating an illustrative embodiment of another pressure control mode for the negative-pressure therapy systems of FIGS. 1 and 2 wherein the x-axis represents time in minutes (min) and/or seconds (sec) and the y-axis represents pressure generated by a pump in Torr (mmHg) that varies with time in a dynamic pressure mode for applying negative pressure in the therapy system;

FIGS. 4 and 5 are schematic diagrams illustrating additional details of a fluid conductor or conduit that may be associated with some example embodiments of the conduits of FIGS. 1 and 2, including some embodiments of conduits that are separate flow channels or lumens of a single fluid conductor such as, for example, a single tube;

FIG. 6 is a perspective view of the topside (closed side) of an embodiment of the dressing interface of FIG. 2 including the reduced pressure delivery tube of FIG. 5 adapted to fluidly couple a reduced pressure treatment device to the dressing interface for providing reduced pressure to a tissue site according to an embodiment of the present invention;

FIG. 7 is a perspective view of the underside (open side) of the dressing interface of FIG. 6 including a single pressure sensing compartment according to an embodiment of the present invention;

FIG. 8 is a perspective end view into the connector associated with the underside (open side) of the dressing interface of FIG. 7 exposing the inside of the single pressure sensing compartment;

FIG. 9 is a perspective view of the underside (open side) of the dressing interface of FIG. 6 including dual pressure sensing compartments according to an embodiment of the present invention;

FIG. 10 is a perspective end view into the connector associated with the underside (open side) of the dressing interface of FIG. 9 exposing the inside of the dual pressure sensing compartment; and

FIG. 11 is a graph illustrating pump pressure (PP), TRAC pressure (TP), and wound pressure (WP) for a first dressing interface A and a second dressing interface B measured over an entire day.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

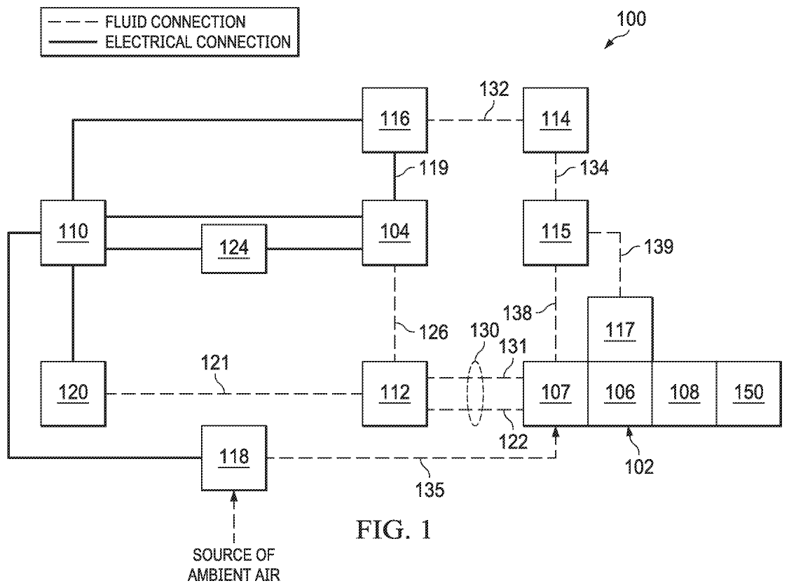

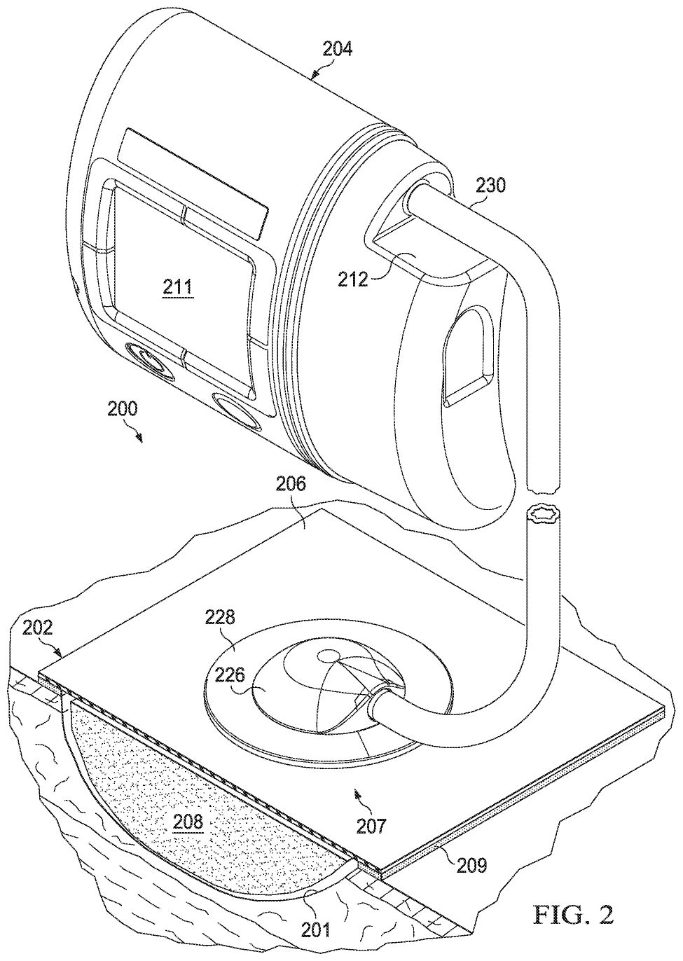

The present technology also provides negative pressure therapy devices and systems, and methods of treatment using such systems with antimicrobial solutions. FIG. 1 is a simplified functional block diagram of an example embodiment of a therapy system 100 that can provide negative-pressure therapy with instillation of treatment solutions to a tissue site 101 in accordance with this specification. FIG. 2 is a schematic diagram, in perspective view with a portion in cross-section, of another example embodiment of a therapy system 200 that can provide negative-pressure therapy without to a tissue site 201 in accordance with this specification incorporating some of the functionality of the therapy system of FIG. 1 that is referred to by similar reference numerals in the 200 series of reference numerals. The therapy system 100 may include a negative-pressure supply, and may include or be configured to be coupled to a distribution component, such as a dressing. In general, a distribution component may refer to any complementary or ancillary component configured to be fluidly coupled to a negative-pressure supply between a negative-pressure supply and a tissue site. A distribution component is preferably detachable, and may be disposable, reusable, or recyclable. For example, a dressing 102 such as dressing 202 is illustrative of a distribution component that may be coupled to a negative-pressure source and other components. The therapy system 100 may be packaged as a single, integrated unit such as a therapy system including all of the components shown in FIG. 1 that are fluidly coupled to the dressing 102. The therapy system may be, for example, a V.A.C. Ulta™ System available from Kinetic Concepts, Inc. of San Antonio, Texas.

The dressing 102 may be fluidly coupled to a negative-pressure source 104 such as negative-pressure treatment device 204. A dressing may include a cover, a tissue interface, or both in some embodiments. The dressing 102, for example, may include cover 106 such as sealing member 206, a dressing interface 107 such as dressing interface 207, and a tissue interface 108 such as manifold 208. A computer or a controller device, such as a controller 110, may also be coupled to the negative-pressure source 104 that may be disposed in the negative-pressure treatment device 204, and may be coupled to a user interface 211. In some embodiments, the cover 106 may be configured to cover the tissue interface 108 and the tissue site, and may be adapted to seal the tissue interface and create a therapeutic environment proximate to a tissue site for maintaining a negative pressure at the tissue site. In some embodiments, the dressing interface 107 may be configured to fluidly couple the negative-pressure source 104 to the therapeutic environment of the dressing. The therapy system 100 may optionally include a fluid container 112, such as canister 212, fluidly coupled to the dressing 102 and to the negative-pressure source 104.

The therapy system 100 may also include a source of instillation solution, such as a solution source 114. A distribution component may be fluidly coupled to a fluid path between a solution source and a tissue site in some embodiments. For example, an instillation pump 116 may be coupled to the solution source 114, as illustrated in the example embodiment of FIG. 1. The instillation pump 116 may also be fluidly coupled to the negative-pressure source 104 such as, for example, by a fluid conductor 119. In some embodiments, the instillation pump 116 may be directly coupled to the negative-pressure source 104, as illustrated in FIG. 1, but may be indirectly coupled to the negative-pressure source 104 through other distribution components in some embodiments. For example, in some embodiments, the instillation pump 116 may be fluidly coupled to the negative-pressure source 104 through the dressing 102. In some embodiments, the instillation pump 116 and the negative-pressure source 104 may be fluidly coupled to two different locations on the tissue interface 108 by two different dressing interfaces. For example, the negative-pressure source 104 may be fluidly coupled to the dressing interface 107 while the instillation pump 116 may be fluidly to the coupled to dressing interface 107 or a second dressing interface 117. In some other embodiments, the instillation pump 116 and the negative-pressure source 104 may be fluidly coupled to two different tissue interfaces by two different dressing interfaces, one dressing interface for each tissue interface (not shown).

The therapy system 100 also may include sensors to measure operating parameters and provide feedback signals to the controller 110 indicative of the operating parameters properties of fluids extracted from a tissue site. As illustrated in FIG. 1, for example, the therapy system 100 may include a pressure sensor 120, an electric sensor 124, or both, coupled to the controller 110. The pressure sensor 120 may be fluidly coupled or configured to be fluidly coupled to a distribution component such as, for example, the negative-pressure source 104 either directly or indirectly through the container 112. The pressure sensor 120 may be configured to measure pressure being generated by the negative-pressure source 104, i.e., the pump pressure (PP). The electric sensor 124 also may be coupled to the negative-pressure source 104 to measure the pump pressure (PP). In some example embodiments, the electric sensor 124 may be fluidly coupled proximate the output of the negative-pressure source 104 to directly measure the pump pressure (PP). In other example embodiments, the electric sensor 124 may be electrically coupled to the negative-pressure source 104 to measure the changes in the current in order to determine the pump pressure (PP).

Distribution components may be fluidly coupled to each other to provide a distribution system for transferring fluids (i.e., liquid and/or gas). For example, a distribution system may include various combinations of fluid conductors and fittings to facilitate fluid coupling. A fluid conductor generally includes any structure with one or more lumens adapted to convey a fluid between two ends, such as a tube, pipe, hose, or conduit. Typically, a fluid conductor is an elongated, cylindrical structure with some flexibility, but the geometry and rigidity may vary. Some fluid conductors may be molded into or otherwise integrally combined with other components. A fitting can be used to mechanically and fluidly couple components to each other. For example, a fitting may comprise a projection and an aperture. The projection may be configured to be inserted into a fluid conductor so that the aperture aligns with a lumen of the fluid conductor. A valve is a type of fitting that can be used to control fluid flow. For example, a check valve can be used to substantially prevent return flow. A port is another example of a fitting. A port may also have a projection, which may be threaded, flared, tapered, barbed, or otherwise configured to provide a fluid seal when coupled to a component.

In some embodiments, distribution components may also be coupled by virtue of physical proximity, being integral to a single structure, or being formed from the same piece of material. Coupling may also include mechanical, thermal, electrical, or chemical coupling (such as a chemical bond) in some contexts. For example, a tube 130 such as tube 230 may mechanically and fluidly couple the dressing 102 to the container 112 in some embodiments. In general, components of the therapy system 100 may be coupled directly or indirectly. For example, the negative-pressure source 104 may be directly coupled to the controller 110, and may be indirectly coupled to the dressing interface 107 through the container 112 by negative-pressure conduit 126 and negative-pressure conduit 131. The pressure sensor 120 may be fluidly coupled to the dressing 102 directly (not shown) or indirectly by pressure-sensing conduit 121 and pressure-sensing conduit 122. Additionally, the instillation pump 116 may be coupled indirectly to the dressing interface 107 through the solution source 114 and the instillation regulator 115 by fluid conductors 132, 134 and 138. Alternatively, the instillation pump 116 may be coupled indirectly to the second dressing interface 117 through the solution source 114 and the instillation regulator 115 by fluid conductors 132, 134 and 139.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” generally refers to a pressure less than a local ambient pressure, such as the ambient pressure in a local environment external to a sealed therapeutic environment provided by the dressing 102. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −75 mm Hg (−9.9 kPa) and −300 mm Hg (−39.9 kPa).

A negative-pressure supply, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 104 may be combined with the controller 110 and other components into a therapy unit. A negative-pressure supply may also have one or more supply ports configured to facilitate coupling and de-coupling the negative-pressure supply to one or more distribution components.

The tissue interface 108 can be generally adapted to contact a tissue site. The tissue interface 108 may be partially or fully in contact with the tissue site. If the tissue site is a wound, for example, the tissue interface 108 may partially or completely fill the wound, or may be placed over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites. Moreover, any or all of the surfaces of the tissue interface 108 may have projections or an uneven, course, or jagged profile that can induce strains and stresses on a tissue site, which can promote granulation at the tissue site.

In some embodiments, the tissue interface 108 may be a manifold such as manifold 208. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam manifold may vary according to needs of a prescribed therapy. For example, in some embodiments, the tissue interface 108 may be a foam manifold having pore sizes in a range of 400-600 microns. The tensile strength of the tissue interface 108 may also vary according to needs of a prescribed therapy. For example, the tensile strength of a foam may be increased for instillation of topical treatment solutions. In one non-limiting example, the tissue interface 108 may be an open-cell, reticulated polyurethane foam such as GranuFoam® dressing or VeraFlo® foam, both available from Kinetic Concepts, Inc. of San Antonio, Texas.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as V.A.C. WhiteFoam® dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 106 may provide a bacterial barrier and protection from physical trauma. The cover 106 may also be constructed from a material that can reduce evaporative losses and provide a fluid seal between two components or two environments, such as between a therapeutic environment and a local external environment. The cover 106 may be, for example, an elastomeric film or membrane that can provide a seal adequate to maintain a negative pressure at a tissue site for a given negative-pressure source. The cover 106 may have a high moisture-vapor transmission rate (MVTR) in some applications. For example, the MVTR may be at least 300 g/m\hat{ }2 per twenty-four hours in some embodiments. In some example embodiments, the cover 106 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. In some embodiments, the cover may be a drape such as drape 206.

An attachment device may be used to attach the cover 106 to an attachment surface, such as undamaged epidermis, a gasket, or another cover. The attachment device may take many forms. For example, an attachment device may be a medically-acceptable, pressure-sensitive adhesive that extends about a periphery such as adhesive 209, a portion, or an entire sealing member. In some embodiments, for example, some or all of the cover 106 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In some embodiments, the dressing interface 107 may facilitate coupling the negative-pressure source 104 to the dressing 102. The negative pressure provided by the negative-pressure source 104 may be delivered through the conduit 131 to a negative-pressure interface, which may include an elbow portion. In one illustrative embodiment, the negative-pressure interface may be a T.R.A.C.® Pad or Sensa T.R.A.C.® Pad available from KCI of San Antonio, Texas. The negative-pressure interface enables the negative pressure to be delivered through the cover 106 and to the tissue interface 108 and the tissue site. In this illustrative, non-limiting embodiment, the elbow portion may extend through the cover 106 to the tissue interface 108, but numerous arrangements are possible.

A controller, such as the controller 110, may be a microprocessor or computer programmed to operate one or more components of the therapy system 100, such as the negative-pressure source 104. In some embodiments, for example, the controller 110 may be a microcontroller, which generally comprises an integrated circuit containing a processor core and a memory programmed to directly or indirectly control one or more operating parameters of the therapy system 100. Operating parameters may include the power applied to the negative-pressure source 104, the pressure generated by the negative-pressure source 104, or the pressure distributed to the tissue interface 108, for example. The controller 110 is also preferably configured to receive one or more input signals, such as a feedback signal, and programmed to modify one or more operating parameters based on the input signals.

Sensors, such as the pressure sensor 120 or the electric sensor 124, are generally known in the art as any apparatus operable to detect or measure a physical phenomenon or property, and generally provide a signal indicative of the phenomenon or property that is detected or measured. For example, the pressure sensor 120 and the electric sensor 124 may be configured to measure one or more operating parameters of the therapy system 100. In some embodiments, the pressure sensor 120 may be a transducer configured to measure pressure in a pneumatic pathway and convert the measurement to a signal indicative of the pressure measured. In some embodiments, for example, the pressure sensor 120 may be a piezoresistive strain gauge. The electric sensor 124 may optionally measure operating parameters of the negative-pressure source 104, such as the voltage or current, in some embodiments. Preferably, the signals from the pressure sensor 120 and the electric sensor 124 are suitable as an input signal to the controller 110, but some signal conditioning may be appropriate in some embodiments. For example, the signal may need to be filtered or amplified before it can be processed by the controller 110. Typically, the signal is an electrical signal that is transmitted and/or received on by wire or wireless means, but may be represented in other forms, such as an optical signal.

The solution source 114 is representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions. Examples of such other therapeutic solutions that may be suitable for some prescriptions include hypochlorite-based solutions, silver nitrate (0.5%), sulfur-based solutions, biguanides, cationic solutions, and isotonic solutions. In one illustrative embodiment, the solution source 114 may include a storage component for the solution and a separate cassette for holding the storage component and delivering the solution to the tissue site 101, such as a V.A.C. VeraLink™ Cassette available from Kinetic Concepts, Inc. of San Antonio, Texas.

The container 112 may also be representative of a container, canister, pouch, or other storage component, which can be used to collect and manage exudates and other fluids withdrawn from a tissue site. In many environments, a rigid container such as, for example, a container 162, may be preferred or required for collecting, storing, and disposing of fluids. In other environments, fluids may be properly disposed of without rigid container storage, and a re-usable container could reduce waste and costs associated with negative-pressure therapy. In some embodiments, the container 112 may comprise a canister having a collection chamber, a first inlet fluidly coupled to the collection chamber and a first outlet fluidly coupled to the collection chamber and adapted to receive negative pressure from a source of negative pressure. In some embodiments, a first fluid conductor may comprise a first member such as, for example, the conduit 131 fluidly coupled between the first inlet and the tissue interface 108 by the negative-pressure interface described above, and a second member such as, for example, the conduit 126 fluidly coupled between the first outlet and a source of negative pressure whereby the first conductor is adapted to provide negative pressure within the collection chamber to the tissue site.

The therapy system 100 may also comprise a flow regulator such as, for example, a regulator 118 fluidly coupled to a source of ambient air to provide a controlled or managed flow of ambient air to the sealed therapeutic environment provided by the dressing 102 and ultimately the tissue site. In some embodiments, the regulator 118 may control the flow of ambient fluid to purge fluids and exudates from the sealed therapeutic environment. In some embodiments, the regulator 118 may be fluidly coupled by a fluid conductor or vent conduit 135 through the dressing interface 107 to the tissue interface 108. The regulator 118 may be configured to fluidly couple the tissue interface 108 to a source of ambient air as indicated by a dashed arrow. In some embodiments, the regulator 118 may be disposed within the therapy system 100 rather than being proximate to the dressing 102 so that the air flowing through the regulator 118 is less susceptible to accidental blockage during use. In such embodiments, the regulator 118 may be positioned proximate the container 112 and/or proximate a source of ambient air where the regulator 118 is less likely to be blocked during usage.

In operation, the tissue interface 108 may be placed within, over, on, or otherwise proximate a tissue site, such as tissue site 101. The cover 106 may be placed over the tissue interface 108 and sealed to an attachment surface near the tissue site 101. For example, the cover 106 may be sealed to undamaged epidermis peripheral to a tissue site. Thus, the dressing 102 can provide a sealed therapeutic environment proximate to a tissue site, substantially isolated from the external environment, and the negative-pressure source 104 can reduce the pressure in the sealed therapeutic environment. Negative pressure applied across the tissue site through the tissue interface 108 in the sealed therapeutic environment can induce macrostrain and microstrain in the tissue site, as well as remove exudates and other fluids from the tissue site, which can be collected in container 112.

In one embodiment, the controller 110 may receive and process data, such as data related to the pressure distributed to the tissue interface 108 from the pressure sensor 120. The controller 110 may also control the operation of one or more components of therapy system 100 to manage the pressure distributed to the tissue interface 108 for application to the wound at the tissue site 101, which may also be referred to as the wound pressure (WP). In one embodiment, controller 110 may include an input for receiving a desired target pressure (TP) set by a clinician or other user and may be programmed for processing data relating to the setting and inputting of the target pressure (TP) to be applied to the tissue site 101. In one example embodiment, the target pressure (TP) may be a fixed pressure value determined by a user/caregiver as the reduced pressure target desired for therapy at the tissue site 101 and then provided as input to the controller 110. The user may be a nurse or a doctor or other approved clinician who prescribes the desired negative pressure to which the tissue site 101 should be applied. The desired negative pressure may vary from tissue site to tissue site based on the type of tissue forming the tissue site 101, the type of injury or wound (if any), the medical condition of the patient, and the preference of the attending physician. After selecting the desired target pressure (TP), the negative-pressure source 104 is controlled to achieve the target pressure (TP) desired for application to the tissue site 101.

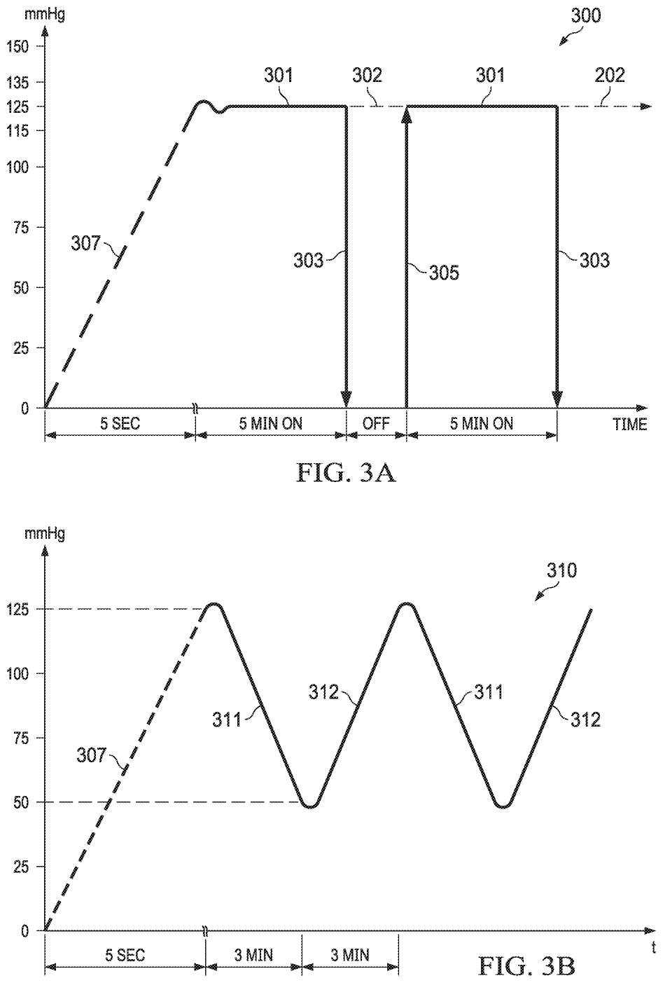

Referring more specifically to FIG. 3A, a graph illustrating an illustrative embodiment of pressure control modes 200 that may be used for the negative-pressure therapy systems of FIGS. 1 and 2 is shown wherein the x-axis represents time in minutes (min) and/or seconds (sec) and the y-axis represents pressure generated by a pump in Torr (mmHg) that varies with time in a continuous pressure mode and an intermittent pressure mode that may be used for applying negative pressure in the therapy system. The target pressure (TP) may be set by the user in a continuous pressure mode as indicated by solid line 301 and dotted line 302 wherein the wound pressure (WP) is applied to the tissue site 101 until the user deactivates the negative-pressure source 104. The target pressure (TP) may also be set by the user in an intermittent pressure mode as indicated by solid lines 301, 303 and 305 wherein the wound pressure (WP) is cycled between the target pressure (TP) and atmospheric pressure. For example, the target pressure (TP) may be set by the user at a value of 125 mmHg for a specified period of time (e.g., 5 min) followed by the therapy being turned off for a specified period of time (e.g., 2 min) as indicated by the gap between the solid lines 303 and 305 by venting the tissue site 101 to the atmosphere, and then repeating the cycle by turning the therapy back on as indicated by solid line 305 which consequently forms a square wave pattern between the target pressure (TP) level and atmospheric pressure. In some embodiments, the ratio of the “on-time” to the “off-time” or the total “cycle time” may be referred to as a pump duty cycle (PD).

In some example embodiments, the decrease in the wound pressure (WP) at the tissue site 101 from ambient pressure to the target pressure (TP) is not instantaneous, but rather gradual depending on the type of therapy equipment and dressing being used for the particular therapy treatment. For example, the negative-pressure source 104 and the dressing 102 may have an initial rise time as indicated by the dashed line 307 that may vary depending on the type of dressing and therapy equipment being used. For example, the initial rise time for one therapy system may be in the range between about 20-30 mmHg/second or, more specifically, equal to about 25 mmHg/second, and in the range between about 5-10 mmHg/second for another therapy system. When the therapy system 100 is operating in the intermittent mode, the repeating rise time as indicated by the solid line 305 may be a value substantially equal to the initial rise time as indicated by the dashed line 307.

The target pressure may also be a variable target pressure (VTP) controlled or determined by controller 110 that varies in a dynamic pressure mode. For example, the variable target pressure (VTP) may vary between a maximum and minimum pressure value that may be set as an input determined by a user as the range of negative pressures desired for therapy at the tissue site 101. The variable target pressure (VTP) may also be processed and controlled by controller 110 that varies the target pressure (TP) according to a predetermined waveform such as, for example, a sine waveform or a saw-tooth waveform or a triangular waveform, that may be set as an input by a user as the predetermined or time-varying reduced pressures desired for therapy at the tissue site 101.

Referring more specifically to FIG. 3B, a graph illustrating an illustrative embodiment of another pressure control mode 310 for the negative-pressure therapy systems of FIGS. 1 and 2 is shown wherein the x-axis represents time in minutes (min) and/or seconds (sec) and the y-axis represents pressure generated by a pump in Torr (mmHg) that varies with time in a dynamic pressure mode that may be used for applying negative pressure in the therapy system. For example, the variable target pressure (VTP) may be a reduced pressure that provides an effective treatment by applying reduced pressure to tissue site 101 in the form of a triangular waveform varying between a minimum and maximum pressure of 50-125 mmHg with a rise time 312 set at a rate of +25 mmHg/minute and a descent time 311 set at −25 mmHg/minute, respectively. In another embodiment of the therapy system 100, the variable target pressure (VTP) may be a reduced pressure that applies reduced pressure to tissue site 101 in the form of a triangular waveform varying between 25-125 mmHg with a rise time 312 set at a rate of +30 mmHg/min and a descent time 311 set at −30 mmHg/min. Again, the type of system and tissue site determines the type of reduced pressure therapy to be used.

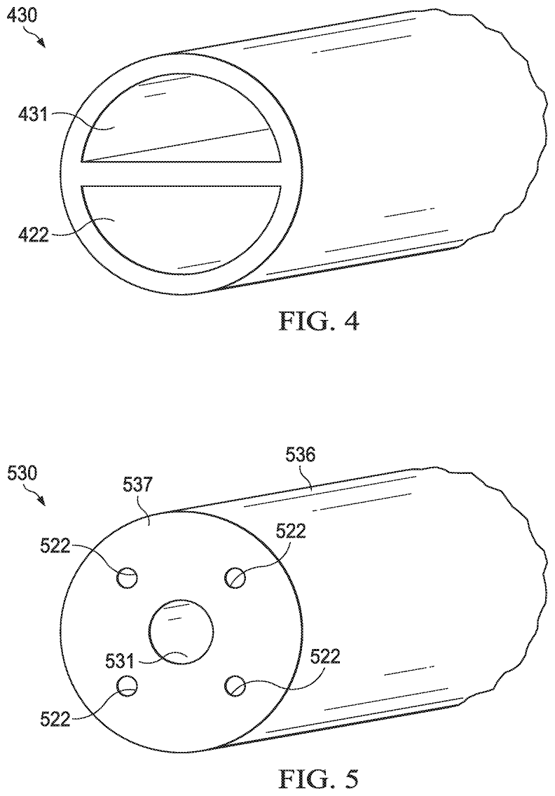

As indicated above, the pressure-sensing conduit 122 and the negative-pressure conduit 131 may be separate lumens disposed in a single fluid conductor such as the tube 130 or the tube 230. Referring to FIG. 4, for example, the pressure-sensing conduit 122 and the negative-pressure conduit 131 may be implemented in a single multi-lumen tube 430 comprising two lumens including lumens 422 and 431 that may correspond to conduit 122 and conduit 131, respectively. When negative pressure is applied, exudates and other fluids are drawn from the tissue site into the canister 112 through the lumen 431 and the pressure sensor 120 can sense the pressure at the tissue site either directly (not shown) or indirectly through conduit 121 and the lumen 422.

In yet another embodiment shown in FIG. 5, the pressure-sensing conduit 122 and the negative-pressure conduit 131 may be disposed in a single multi-lumen tube 530 having an outside surface 536 generally tubular in shape and terminating in an end surface 537. The tube 530 in this embodiment comprises a central lumen 531 and a plurality of peripheral lumens 531 corresponding to conduit 122 and conduit 131, respectively. When negative pressure is applied, exudates and other fluids are drawn from the tissue site into the canister 112 through the central lumen 531 which may have a larger diameter than the peripheral lumens 522 to accommodate the volume of exudates and other fluids, and the pressure sensor 120 can sense the pressure at the tissue site either directly (not shown) or indirectly through conduit 121 and the lumens 522. In another embodiment wherein the regulator 118 is fluidly coupled directly to the dressing 102 by a third conduit 135, the third conduit may also be one or more of the peripheral lumens 522 disposed in the tube 530 so that ambient air may be drawn into the tissue interface 108 from regulator 118 through the peripheral lumens 522.

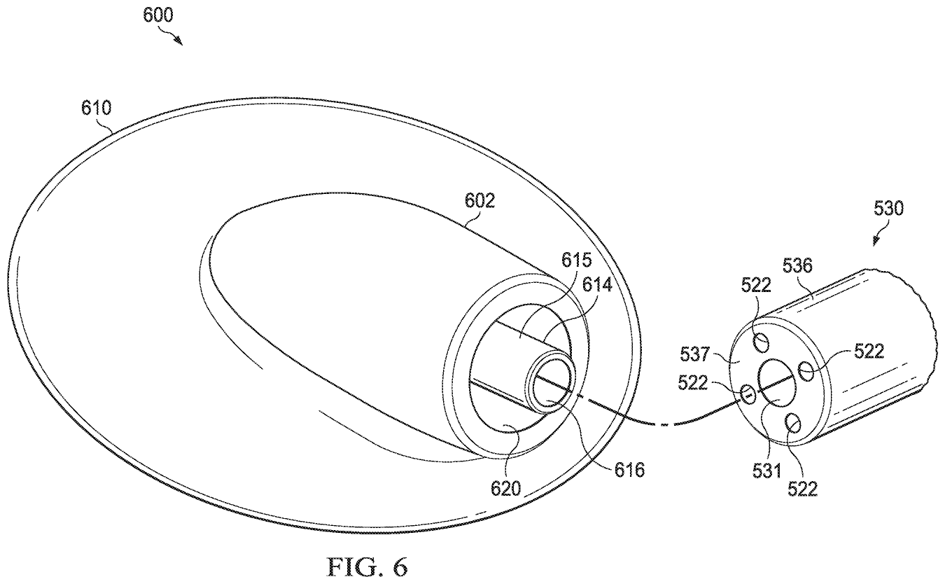

Referring to FIGS. 6-8, an example embodiment of the dressing interface 107 and/or dressing interface 207 may be a dressing interface 600 including a reduced pressure delivery tube such as the multi-lumen tube 530 that is adapted to fluidly couple a reduced pressure treatment device such as the reduced pertinent device 204 to the dressing interface 600 for providing reduced pressure to a tissue site. In some example embodiments, the dressing interface 600 functions in a fashion similar to the dressing interface 107 as described above. The dressing interface 600 comprises a conduit housing 602 that may be similar to the conduit housing 226 of the dressing interface 207. The conduit housing 602 may comprise a recessed compartment 604 defining a cavity having an opening 606 adapted to be in fluid communication with the tissue site when the conduit housing 602 is positioned on the tissue site. The conduit housing 602 may further comprise a base 610 that may be similar to the base 228 of the dressing interface 207. The base 610 may have an aperture 613 surrounding the opening of the recessed compartment 604 and a manifold-contacting surface 612 extending from the aperture 613. In some embodiments, the dressing interface 600 further comprises a reduced-pressure interface 614 having an outside surface 615 and a reduced-pressure lumen 616 with one end extending through the conduit housing 602 to a reduced pressure port 618 that is fluidly coupled to the recessed compartment 604. The other end of the reduced-pressure lumen 616 is adapted to be coupled through the central lumen 531 to a reduced-pressure source such as the reduced-pressure source 104 described above.

The dressing interface 600 also may comprise a pressure-detection port 620 extending through the conduit housing 602 and adapted to be fluidly coupled through the peripheral lumens 522 to a pressure sensor such as the pressure sensor 120, and a pressure-detection chamber 622 having an inside surface 623 forming a cavity fluidly coupled to the pressure-detection port 620. The cavity of the pressure-detection chamber 622 is formed by its inside surface 623, the outside surface 615 of the reduced-pressure interface 614, and the end surface 537 of the tube 530. The cavity of the pressure-detection chamber 622 opens to an orifice 624 disposed within and fluidly coupled to the cavity of the recessed compartment 604. Thus, when the tube 530 is assembled with the conduit housing 602, the central lumen 531 fits over the reduced-pressure interface 614 in fluid communication with the reduced-pressure lumen 616, and the peripheral lumens 522 are fluidly coupled to the cavity of the pressure-detection chamber 622. The dressing interface 600 also may comprise one or more standoffs such as, for example, standoff 626 disposed within the pressure-detection chamber 622 to prevent the end surface 537 of the tube 530 butting up against the inside surface 631 of the liquid-air filter 630 and collapsing the cavity of the pressure-detection chamber 622.

The dressing interface 600 may further comprise a liquid-air separator 630 such as, for example, a hydrophobic filter, having an external surface 629 and an internal surface 631 that seals and closes the orifice 624 to limit flow of liquids into the pressure-detection chamber 622. The purpose of the liquid-air separator 630 is to prevent the peripheral lumens 522 from being clogged by liquids and exudates drawn out of the tissue interface 108 by the negative pressure being applied to tissue site. In some example embodiments, the hydraulic filter may comprise PTFE (poly-tetra-fluoro-ethylene) woven fabric such as, for example, a woven fiberglass membrane coated with PTFE. In other example embodiments, the PTFE woven fabric may comprise a woven base cloth that may comprise warp threads running the length of the hydrophobic filter and weft threads running across the width of the filter. However, such materials may abrade during production or operation of the dressing interface 600, especially along the edges of the orifice 624. In areas where the PTFE woven fabric has not been ablated or destroyed by abrading, that portion is still viable as a liquid-air separator to prevent liquids and exudates from clogging the peripheral lumens 522 thereby protecting the integrity of the pressure sensor 120. Therefore, it would be desirable to minimize the occurrences of the PTFE woven fabric abrading along the edges of the orifice 624.

In some example embodiments, the dressing interface 600 may further comprise adhesive 636 disposed between the edges of the internal surface 631 of the liquid-air separator 630 and the edge of the orifice 624 to reduce the extent to which the assembly or operation of the dressing interface 600 might ablate the liquid-air separator 630 along the edges of the orifice 624. In some example embodiments, the dressing interface 600 may further comprise adhesive 638 disposed over the edges of the external surface 629 of the liquid-air separator 630 and the edge ofthe orifice 624 to further reduce the extent to which the assembly or operation of the dressing interface 600 might ablate the liquid-air separator 630. In either embodiment, the adhesive 636 and/or the adhesive 638 they cover the outer edge of the liquid-air separator 630 between the internal surface 631 and the external surface 629.

The adhesive 636 and/or the adhesive 638 may comprise different types of material applied by different methods. In one example embodiment, the adhesive 636 disposed between the edges of the internal surface 631 of the liquid-air separator 630 and the edge of the orifice 624 may comprise an adhesive gel curable by UV light. In some embodiments, a bead of the adhesive gel may be applied to the edges of orifice 624 and the PTFE woven fabric may be positioned or placed or placed on edges of orifice 624. In some embodiments, a slight pressure may be applied to the PTFE woven fabric at the same time so that the woven fabric to pushes into the adhesive gel. Preferably, the edges of the PTFE woven fabric may also be contained within the bead of the adhesive gel so as not to be exposed to the liquids and exudates flowing in the recessed compartment 604. The adhesive gel may then be cured with high intensity UV light so that the adhesive bead retains a three-dimensional structure and/or is semi-rigid when cured by the UV light. This encapsulation process in some embodiments may use an adhesive material supplied by Dymax, number DYM 3099, or any other adhesive gel that is stable, good for gap-filling, and/or retains a three-dimensional structure when cured.

In another example embodiment, the adhesive 638 disposed over the edges of the external surface 629 of the liquid-air separator 630 and the edge of the orifice 624 also comprises an adhesive gel curable by UV light. In some embodiments, a second bead of the same adhesive gel may be applied to the edges of the PTFE woven fabric over the orifice 624 thus encapsulating the external and outer edge of the woven fabric with the adhesive material. The adhesive gel also may be a type that is cured with high intensity UV light. The adhesive gel may then be cured with high intensity UV light so that the adhesive bead retains a three-dimensional structure and/or is semi-rigid when cured by the UV light.

As a result of using one or both of the embodiments described above, the liquid-air separator 630 is assembled to cover the orifice 624 without being affected by forces or temperatures associated with the manufacturing process of the dressing interface 600 that may degrade bonding between the edges of the PTFE woven fabric and the edges of the orifice 624. Preventing ablation of the liquid-air separator 630 prevents liquids and exudates from seeping into the cavity of the pressure detection chamber 622 to reduce the possibility that peripheral lumens 522 become clogged thereby protecting the integrity of the pressure sensor 120 to accurately detect the wound pressure (WP) as described above.

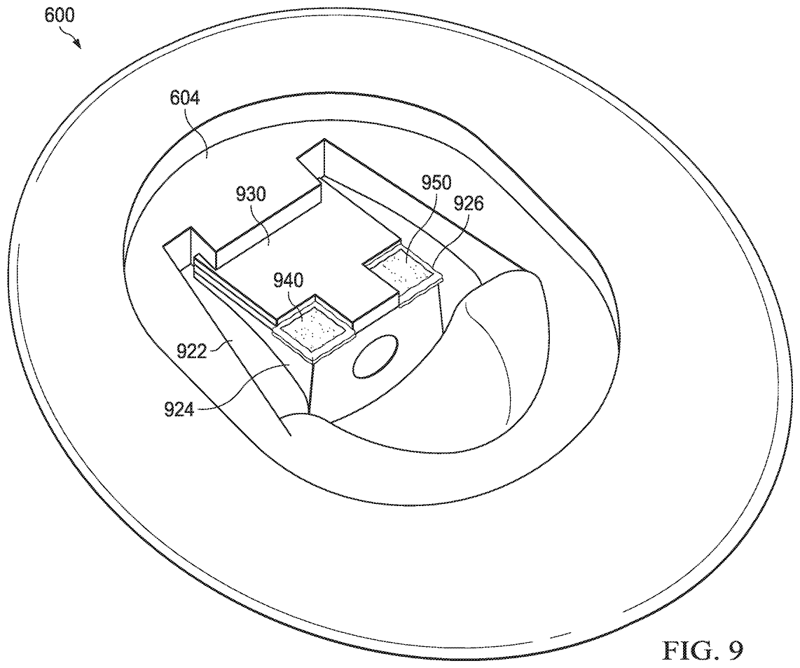

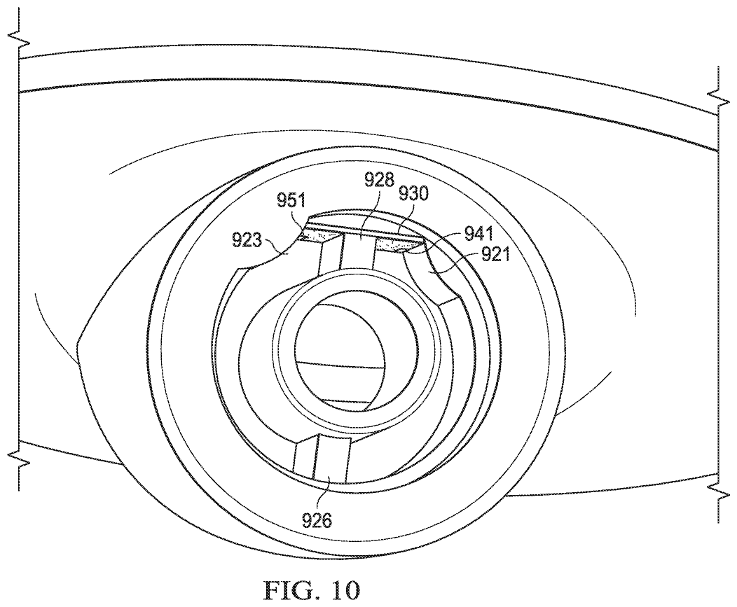

Referring to FIGS. 9 and 10, the dressing a 600 may comprise another example embodiment of a pressure-detection chamber such as, for example, a dual pressure-detection chamber 922 divided into two cavities by standoffs 926 and 928 to form two inside surfaces 921 and 923 including separate orifices 924 and 926 disposed in the recessed compartment 604. The two orifices 924 and 926 may be spaced apart as shown with the remaining portion of the pressure-detection chamber 922 being covered by an upper wall 930. Each of the two orifices 924 and 926 may be covered by liquid-air separators such as hydrophobic filters 940 and 950 of the types described above that have internal surfaces 941 and 951, respectively. The hydrophobic filters 940 and 950 may each be sealed to the edges of the orifices 924 and 926 by the curable adhesive gels as described above. Thus, when the tube 530 is assembled with the conduit housing 602, the central lumen 531 fits over the reduced-pressure interface 614 in fluid communication with the reduced-pressure lumen 616. Additionally, the two cavities of the pressure-detection chamber 922 are formed by the inside surfaces 921 and 923, the outside surface 615 of the reduced-pressure interface 614, and the end surface 537 of the tube 530 so that two of the peripheral lumens 522 are fluidly coupled to each of the cavities of the dual pressure-detection chamber 922. For certain therapy treatments, it is desirable to have two filters such as hydrophobic filters 940 and 950 spaced apart to detect the pressure at different locations along the surface of a tissue interface such as the tissue interface 108 and/or the manifold 208. Preventing ablation of the hydrophobic filters 940 and 950 prevents liquids and exudates from seeping into the cavity of the dual pressure detection chamber 922 to reduce the possibility that peripheral lumens 522 become clogged and protecting the integrity of the pressure sensors.

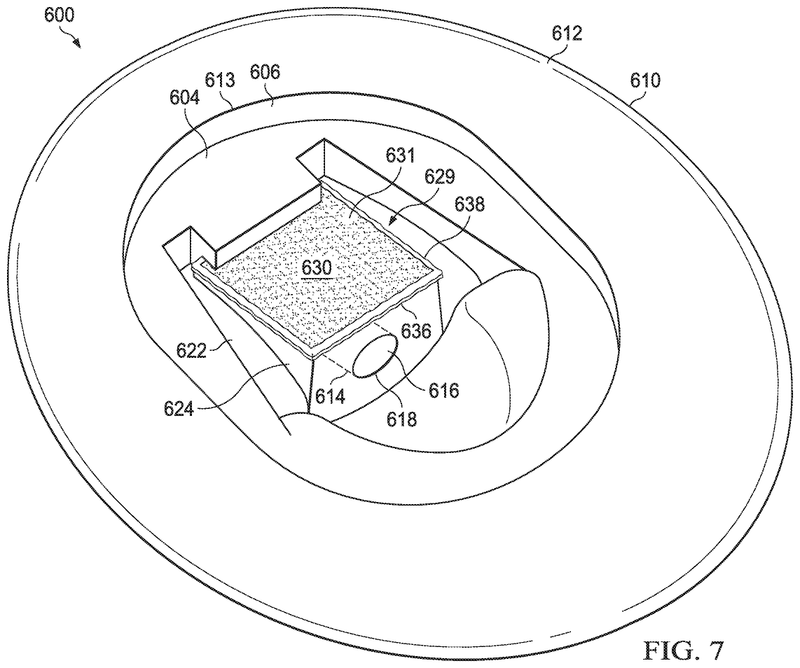

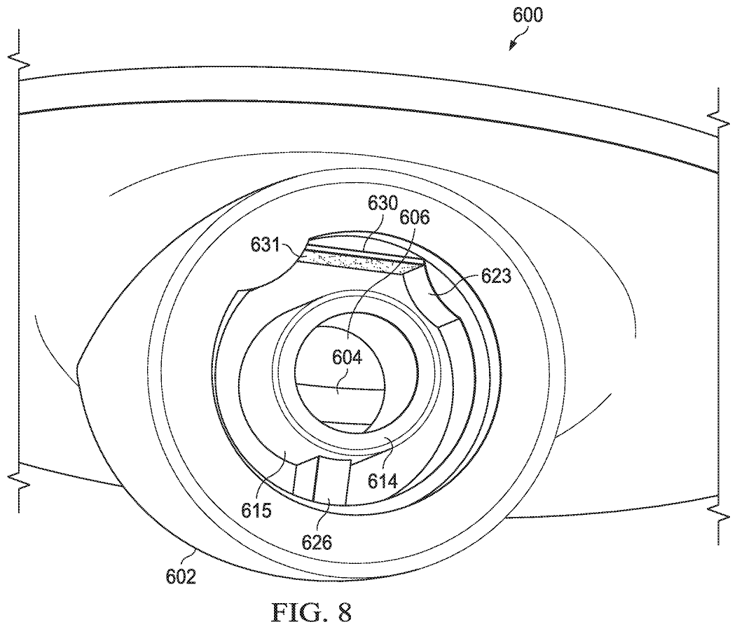

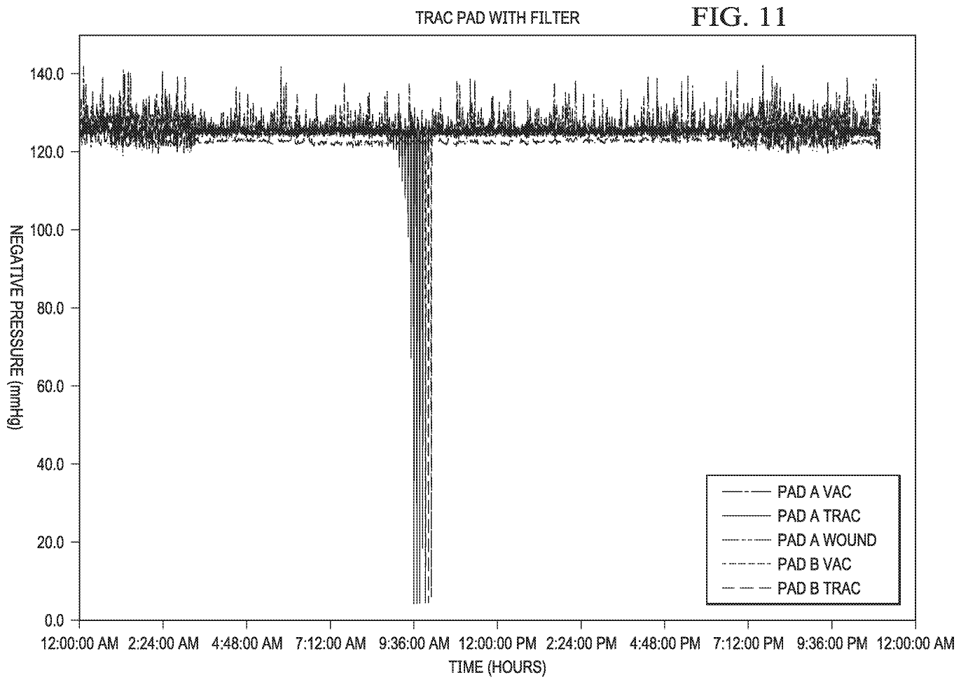

Referring back to FIGS. 7 and 8, testing of the single pressure-detection chamber 622 and the single filter 630 has shown that such liquid-air separators and the method of fabricating them have worked as anticipated. Testing was conducted using a single filter 630 having an area of 7 mm×9 mm (the “single test filter”) and subjecting that filter to a flow rate of about 100 mL/day with a solution comprising wound fluids. The area of the single filter 630 is sufficiently large so that it is not overly restrictive to the flow rate of lumens 522 and the rest of the negative pressure supply system. Referring to FIG. 11, a graph illustrating pump pressure (PP), TRAC pressure (TP), and wound pressure (WP) for a first dressing interface A including the single filter 630 and a second dressing interface B without a hydrophobic filter over an entire day is shown. The TRAC pressure (TP) is the control pressure in the container 112 representing the negative pressure provided by the lumens 522, and the wound pressure (WP) is the negative pressure under the filter 630 that tracks the TRAC pressure (TP). As can be seen in the graph, the pressures associated with dressing interface A remained substantially similar to those pressures associated with dressing interface B, and remained substantially stable within a range of about 120-140 mmHg except for a drop in negative pressure at 9:36 AM resulting from a device failure having nothing to do with the dressing interfaces or the test itself. Thus, dressing interface A including the single filter 630 clearly performed as well as dressing interface B while being able to prevent wound fluids from entering the pressure-detection chamber 622 so that the lumens, such as the lumens 522 of the single multi-lumen tube 530, are not clogged by the wound fluids.

In some example embodiments of the dressing interface 600, the area of the single filter 630 is sufficiently large so that it is not overly restrictive to the flow rate, but in some embodiments may be even smaller as long as the filter it is not overly restrictive to the flow rate. For example, the combined area of the dual filters 940 and 950 is smaller as shown in FIGS. 9 and 10 as long as the combined area is sufficiently large so that the filters will not be the most significant flow restrictions within the system including, for example, flow restrictions created by a hydrophobic filter contained within the canister 212 and/or the diameter of one single lumen 522 remaining open after the others become clogged. One skilled in the art would be able to determine the minimum area of the filters so as not be overly restrictive of the flow rate of the dressing inter face 600 within the system.

In other example embodiments of the dressing interface 600, the area of the filters may be large enough to require web supports for preventing the filters from flexing causing the filters to wear and lose their ability to sufficiently function as a liquid-air separator. The web supporters may be a separate component in some embodiments or may be molded underneath the filter within the pressure-detection chamber 622. In other embodiments, the web supporter may be suspended from the orifice 624 of the pressure-detection chamber 622 prior to the filter being disposed over the orifice and sealed to the orifice with adhesives as described above.

In some example embodiments, the controller 110 may be configured to cause the regulator 118 to provide routine purges of ambient air to the sealed therapeutic environment to clear deposits from the filters 630, 940 and/or 950. Clearing such deposits by purging the therapeutic environment further prevents liquids and exudates from entering the pressure-detection chamber 622 and ultimately the lumens 522 of the single multi-lumen tube 530.

The systems, apparatuses, and methods described herein may provide significant advantages over and above the significant advantages described above. For example, using adhesives to seal the filter within the pressure-detection chamber as described above may facilitate the manufacturing process of the dressing interface 600. If the manufacturing process includes injection molding the dressing interface 600 and positioning of the filter within the recessed compartment 604, the filter may be damaged by the temperature and pressure during injection molding and/or be improperly positioned over the orifice 624 and consequently out of tolerance. The process described above utilizing adhesives obviates the problems that may be associated with the injection molding process.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 102, the container 112, or both may be eliminated or separated from other components for manufacture or sale. In other example configurations, the controller 110 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing interface for connecting a reduced-pressure source to a manifold to treat a tissue site with reduced pressure, the dressing interface comprising:
a conduit housing having a cavity defined by a recessed compartment and an opening;
a base having a manifold-contacting surface and an aperture surrounding the opening;
a reduced-pressure port extending through the conduit housing and fluidly coupled to the recessed compartment, the reduced-pressure port adapted to be coupled to the reduced-pressure source;
a pressure-detection port extending through the conduit housing and adapted to be fluidly coupled to a pressure sensor;
a pressure-detection chamber fluidly coupled to the pressure-detection port and having an orifice disposed within the recessed compartment; and
a liquid-air separator closing the orifice of the pressure-detection chamber to limit the flow of liquids into the pressure-detection chamber.

2. The dressing interface of claim 1, wherein the liquid-air separator is a hydrophobic filter.

3. The dressing interface of claim 1, wherein the liquid-air separator is a hydrophobic filter comprising PTFE.

4. The dressing interface of claim 1, wherein the liquid-air separator is a hydrophobic filter comprising a woven fiberglass membrane coated with PTFE.

5. The dressing interface of claim 1, wherein the liquid-air separator is a hydrophobic filter comprising a woven base cloth.

6. The dressing interface of claim 1, wherein the liquid-air separator is a hydrophobic filter comprising woven base cloth having warp threads and weft threads.

7. The dressing interface of claim 1, wherein the orifice has an edge, the dressing interface further comprising an adhesive disposed between the liquid-air separator and the edge of the orifice.

8. The dressing interface of claim 7, further comprising an adhesive disposed over the liquid-air separator and the edge of orifice.

9. The dressing interface of claim 7, wherein the adhesive comprises an adhesive gel curable by UV light.

10. The dressing interface of claim 1, further comprising a second pressure-detection chamber fluidly coupled to a second pressure-detection port and having an orifice disposed within the recessed compartment; and a second liquid-air separator closing the orifice of the second pressure-detection chamber to limit the flow of liquids into the second pressure detection chamber.

11. A dressing interface for connecting a reduced-pressure source to a manifold to treat a tissue site with reduced pressure, the dressing interface comprising:
a conduit housing having a cavity defined by a recessed compartment and an opening;
a base having a manifold-contacting surface and an aperture surrounding the opening;
a reduced-pressure conduit extending through the conduit housing and fluidly coupled to the recessed compartment;
a pressure-detection conduit extending through the conduit housing and having at least one lumen with an orifice disposed within the recessed compartment; and
a liquid-air separator closing the orifice of the at least one lumen.

12. The dressing interface of claim 11, wherein the liquid-air separator is a hydrophobic filter.

13. The dressing interface of claim 11, wherein the liquid-air separator is disposed within the lumen.

14. The dressing interface of claim 11, wherein the lumen has walls and further comprising an adhesive disposed between the liquid-air separator and the walls of the lumen.

15. The dressing interface of claim 11, wherein the at least one lumen is a first lumen, further comprising a second lumen having an orifice disposed within the recessed compartment; and a second liquid-air separator closing the orifice of the second lumen.

16. A dressing interface for connecting a reduced-pressure source to a manifold to treat a tissue site with reduced pressure, the dressing interface comprising:
a conduit housing having a cavity defined by a recessed compartment and an opening;
a base having a manifold-contacting surface and an aperture surrounding the opening;
a reduced-pressure port extending through the conduit housing and fluidly coupled to the recessed compartment;
a pressure-detection port extending through the conduit housing and forming a chamber having an orifice disposed within the recessed compartment; and
a liquid-air separator closing the orifice of the chamber;
wherein the dressing interface is produced by the steps comprising:
applying a first bead of adhesive gel to the orifice that is curable by UV light;
placing the liquid-air separator on the adhesive gel over the orifice to close the chamber; and
curing the adhesive gel with UV light to form a semi-rigid structure when cured by the UV light.

17. The dressing interface of claim 16, further comprising the steps of applying a second bead of adhesive gel to the liquid-air separator opposite the first bead of adhesive gel over the orifice that is curable by UV light; and curing the second bead of adhesive gel with UV light to form a semi-rigid structure when cured by the UV light.

18. The dressing interface of claim 16, wherein the liquid-air separator is a hydrophobic filter.

19. The dressing interface of claim 16, wherein the liquid-air separator is a hydrophobic filter comprising PTFE.

20. The dressing interface of claim 16, wherein the liquid-air separator is a hydrophobic filter comprising a woven base cloth.

21. (canceled)
